(19) United States
(12) Patent Application Publication
(10) Pub. No.: US 2012/0109151 A1
(43) Pub. Date: May 3, 2012

(54) System and Computer Assisted Surgery

(75) Inventors: Lena Maier-Hein, Heidelberg (DE); Hannes Kenngott, Heidelberg (DE); Hans-Peter Meinzer, Heidelberg (DE); Beat Peter Mueller-Stich, Heidelberg (DE)

(73) Assignees: RUPRECHT-KARLS-UNIVERSITÄT HEIDELBERG, Heidelberg (DE); Deutsches Krebsforschungszentrum Stiftung des öffentlichen Rechts, Heidelberg (DE)

(21) Appl. No.: 13/262,425

(22) PCT Filed: Mar. 4, 2010
(86) PCT No.: PCT/EP2010/001362

§ 371 Date: Jan. 13, 2012

Related U.S. Application Data
(60) Provisional application No. 61/166,370, filed on Apr. 3, 2009.

(51) Int. Cl. A61B 19/00

(52) U.S. Cl. 606/130

(57) ABSTRACT

The present invention relates to a system and method of computer assisted surgery in soft tissue. The system comprises a navigated instrument (14) suitable to be inserted into a living object's soft tissue body part, wherein said navigated instrument (14) when inserted into the body part is suitable to define at least a portion of a section plane with respect to said soft tissue body part. The system further comprises a computer assisted guiding means to assist or enable guiding the instrument (14) upon insertion into the body part such that the section plane defined by said inserted instrument (14) coincides with a planned section plane.

### FIELD OF THE INVENTION

The present invention relates to a system and a method for computer assisted surgery in soft tissue.

### BACKGROUND OF THE INVENTION

Computer based surgical planning gives the surgeon all necessary information for choosing the best therapeutic strategy for every individual patient. For example, research scientists at the German Cancer Research Centre have developed a surgical planning software for liver surgery which extracts all the data from routinely acquired preoperative medical images and delivers optimized models to lay open the details of the visceral anatomy, which highly differs from patient to patient. Also, computer based surgical planning allows for obtaining quantitative information from preoperative medical images, such as the volume of organs or tumors, or the volumes of a portion of a liver to be transplanted and of the portion remaining with the donor. Another quantitative information that can be derived from medical images by modern planning software is the vascular supply of given areas of the organ under treatment. All of this information can be used in predefining the section plane along which the organ is to be cut during the actual surgery. By carefully planning the section plane based on the above information, the chances of successful surgery can be greatly improved.

For example, if a tumor is to be excised from an organ, it must be ensured that the tumor is resected completely, while removing as little healthy tissue as possible. The appropriate section plane ensuring this can be predetermined using computer based surgical planning. To give another example, in liver transplantations, it must be ensured that both the donor and the receiver obtain a sufficient liver volume, sufficient blood supply and drainage. Again, an appropriate section plane allowing this can be predetermined using computer based surgical planning.

In one planning approach, the software may automatically generate resection proposals based on vascular supply areas calculated from annotated vessels. In an alternative approach, the user, i.e. the surgeon planning a surgery, can freely define a section plane through the organ, while using the software to calculate the volumes of the resected and remaining parts. In any case, the outcome is a planned section plane optimized for the given anatomy. More details about planning of section planes can be found in Meinzer H P, Thorn M, Cardenas C, “Computerized Planning of Liver Surgery—an Overview”; Computers & Graphics 2002; 26(4):569-576; and Fischer L, Hoffmann K, Neumann J O, Schöbinger M, Grenacher L, Raeleff B, Friess H, Meinzer H P, Büchler M W, Schmidt J, Schemmer P; “The Impact of Virtual Operation Planning on Liver Surgery”, Imaging Decisions 2007; 1:39-44.

While the section plane can thus be planned with great sophistication, unfortunately it is in practice often difficult to actually cut an organ according to the planned section plane under surgery. This is especially true in cases where important structures that could be helpful for orientation, such as a tumor or vessels, are inside the organ and are therefore occluded, which in the above mentioned example of liver resection is always the case. Accordingly, it requires an enormous amount of skill and experience to carry out the planned cuts under surgery.

A promising tool for assisting carrying out predetermined cuts are computer assisted systems for information and navigation, which have been widely used in surgery during the last ten years. Generally, such systems operate based on a model of the patient generated prior to the surgery, which is registered with the true anatomy of the patient at the beginning of the surgery. For example, optical or magnetic systems for localizing surgical instruments can be used to visualize such instruments in relation to the critical anatomic structures. This works very well and reliably as long as the organs in the target area are stationary as it is often the case in neurosurgery and orthopedics. However, soft tissue organs are subject to constant motions and deformations, caused for example by the breathing cycle or heart beat of the patient, movement of the patient and manipulation by the instrument, such that commercially available navigation systems are not suitable for abdominal surgery. A further problem is the changing topology of the organ, once it has been cut under surgery. All of this makes it rather difficult to employ navigation systems in soft tissue surgery.

A few concepts in the specific context of computer assisted liver resection have been known. One approach is to insert the current position of instruments in 3D-medical data using Augmented Reality, as has been described for calibrated ultra sound apparatuses in Beller S, Hünerbein M, Lange T, Eulenstein S, Gebauer B, Schlag P M; “Image-Guided Surgery of Liver Metastases by Three-Dimensional Ultrasound-based Optoelectronic Navigation”; Br J. Surg. 2007 July; 94(7):866-75, and for CT-apparatuses by Feuerstein M, Mussack T, Heining S M, Navab N; “Intraoperative Laparoscope Augmentation for Port Placement and Resection Planning in Minimally Invasive Liver Resection”; IEEE Trans Med Imaging; 2008 March; 27(3):355-69.

A different navigation scheme based on surface registering is described in D. M. Cash, M. I. Miga, T. K. Sinha, R. L. Galloway, and W. C. Chapman, “Compensating for intraoperative soft-tissue deformations using incomplete surface data and finite elements”, IEEE T Med Imaging, 24(11):1479-1491, November 2005 and in D. M. Cash, M. I. Miga, S. C. Glasgow, B. M. Dawant, L. W. Clements, Z. Cao, R. L. Galloway, and W. C. Chapman; “Concepts and preliminary data toward the realization of image-guided liver surgery”; J Gastrointest Surg, 11(7):844-859, July 2007. In this method, high resolution planning images can be registered with intraoperatively acquired surface data using a tracked and calibrated surface scanner. Then, internal structures can be transformed based on the surface motion using finite element models. However, since this method takes several minutes to conduct, it can only be applied very few times during a single intervention.

So far, no method has been known in which planning data can be reliably associated with the patient during surgery, i.e. after the topology changes due to cuts having been made. Accordingly, once resection has begun, none of the above approaches will provide reliable information anymore.

### SUMMARY OF THE INVENTION

It is an object of the present invention to provide a system and method allowing for precisely and reliably transferring preoperatively generated planning data to the intraoperative situs.

This object is met by an apparatus according to claim 1 and a method according to claim 37. Preferable embodiments are defined in the dependent claims.

According to an embodiment of the present invention, a system for computer assisted surgery in soft tissue comprises an instrument suitable to be inserted into a living object's soft tissue body part, wherein said instrument when inserted into the body part is suitable to define at least a portion of a section plane with respect to said soft tissue body part, and a computer assisted instrument guiding means to assist or enable guiding the instrument upon insertion into the body part such that the section plane defined by said inserted instrument coincides with the planned section plane.

Preferably, the instrument is a navigated instrument. Herein, the expression “navigated instrument” is to be interpreted in a broad sense and shall cover both, instruments that can be tracked using a tracking system as well as instruments guided by a robot. However, the computer assisted guiding means could also be such that they assist guiding of a non-navigated instrument, as will be explained below.

According to the invention, the instrument is an instrument which upon insertion into the body part defines at least a portion of a section plane, i.e. a two-dimensional plane within the soft tissue body part. In other words, a two-dimensional plane can be defined directly within the body part upon insertion of a single instrument only. Then, after the instrument has been inserted, subsequent motion of the soft tissue body part will not affect the section plane as defined by the instrument. Further, since the system comprises computer assisted instrument guiding means, the instrument can be guided to be inserted such that the section plane defined thereby coincides with a planned section plane. Accordingly, the system allows for reliably and easily transferring a planned section plane obtained for example with a surgical planning software as described above to the actual body part under surgery.

Preferably, the instrument comprises a set of elongate members suitable for being simultaneously inserted into the soft tissue body part. The plane defined by the instrument may then be the envelope of the set of elongate members. The elongate members may for example comprise a set of substantially parallel needle-like or blade-like elements. While the elongate members are preferably parallel such as not to compress or stretch the tissue upon insertion, they need not be straight. Instead, the elongate members could be curved and therefore define a curved section plane.

In a preferred embodiment, the instrument is provided with marking means for marking the section plane or portions thereof within the soft tissue body part. In this embodiment, the instrument can be regarded as a marking tool. After the instrument has been used for marking the section plane or portions thereof, the instrument can be removed from the soft tissue body part. Then, even if the soft tissue body part deforms or even changes its topology under surgery, the marking of the section plane as originally defined upon insertion of the instrument remains. Accordingly, the surgeon can simply cut along the marked tissue without paying attention to deformation or change of topology and still be sure to cut along the section plane as defined upon insertion of the instrument.

Preferably the marking means of said instrument comprises one or more of the following: denaturation means for marking the section plane by denaturation, such as cauterization or coagulation, color marking means for marking the section plane by means of applying color to the tissue, or marking object insertion means for inserting marking objects to the tissue, such as needles or threads. With any of these marking means, portions of the tissue spanning the section plane defined upon insertion of the instrument are permanently marked, such that the section plane can be visually recognized during surgery even after removal of the navigated instrument.

A very advantageous marking scheme is based on coagulation or cauterization. In a preferred embodiment, the instrument, when inserted into the soft tissue body part, is suitable for coagulating our cauterising tissue in the vicinity of each elongate member and/or in between adjacent elongate members. Due to coagulation or cauterization, the tissue changes its color, such that a two-dimensional section plane is permanently marked. However, the cauterization or coagulation has the additional advantage that bleeding can be prevented upon subsequent cutting along the section plane. Also, if the instrument should accidentally be inserted incorrectly and hurt a tumor, tumor seeding can be prevented by the coagulation or cauterization in a similar way as known from tumor ablation.

Note that the marking tool is only one example of the navigated or non-navigated instrument of the invention. In an alternative embodiment, the instrument may be a preferably navigated cutting tool suitable for coagulating and/or cauterising the tissue in the vicinity of each elongate member and/or between adjacent elongate members and for cutting tissue between at least some adjacent ones of said elongate members when inserted into the soft tissue body part. Accordingly, this embodiment allows for quasi-automatically cutting the tissue along the section plane defined upon insertion of the instrument into the soft tissue body part and to prevent bleeding and tumor seeding due to coagulation or cauterization. Accordingly, this embodiment allows to greatly facilitate the surgery and reduce the risk for the patient.

Preferably, in addition to the set of elongate members mentioned above, the instrument comprises a further set of blade members adapted for cutting the tissue between said elongate members after insertion into the soft tissue body part. In one embodiment, the instrument may comprise a first and a second member, each of said first and second members comprising a set of substantially parallel blade-like elements, said first and second members being moveable to a first position, in which the blade-like elements of the first member are substantially congruent with the blade-like elements of the second member, and a second position, in which—when viewed from a direction perpendicular to the direction of relative motion of the first and second members—blade-like elements of the first member are located substantially between adjacent blade-like elements of said second member. Such embodiment is very easy in construction and at the same time highly efficient. When the first and second members are in the first position, the instrument can be inserted into the soft tissue body part, since the blade-like elements of the first and second members are substantially congruent with each other, which means that the insertion is similar to a situation where only one of the members were inserted. After insertion, the section plane is physically defined by the elongate members being inserted into the tissue. Immediately after insertion of the instrument with the first and second members in the first position, the tissue surrounding the instrument may be cauterised or coagulated to stop bleeding. Then, by moving the first and second members into the second position, the tissue is cut along the section plane as defined upon insertion of the instrument, which in turn coincides with the planned section plane.

In one embodiment, the instrument is a preferably navigated laparoscopic instrument, a preferred embodiment of which being shown in the description below.

In an alternative embodiment, the instrument is formed by a cutting tool adapted for cutting the tissue between adjacent elongate members by electro surgery.

In yet an alternative embodiment, the instrument is a jig which defines a section plane along which the tissue can be cut while the jig remains inserted into the soft tissue body part. This embodiment is conceptionally similar to the marking tool described above, except that the instrument itself is the marking which remains inserted in the soft tissue body part.

Irrespectively of the specific embodiment of the instrument, be it a marking tool, a cutting tool or a jig, the assisted instrument guiding means may comprise positioning assisting means for assisting a user to position the instrument at the soft tissue body part prior to insertion, wherein said positioning assisting means is adapted to generate and display an image allowing a user to assess how the leading edge of the instrument has to be moved in order to approach the intersection line of the surface of the body part and the planned section plane. Such positioning assisting means greatly facilitates positioning the instrument prior to insertion, such that the section plane defined by said instrument upon insertion will coincide with the planned section plane.

Preferably, the image generated by the positioning assisting means represents a relative position between said intersection line and a projection of said leading edge of the instrument onto the surface of said body part. Herein, the projection may for example be a projection along a vector defining a predetermined insertion direction. Note that the projection of the leading edge of the navigated instrument onto the surface of the body part resembles two-dimensional information only. However, this is exactly the two-dimensional information that is crucial for correctly positioning the instrument prior to insertion. By reducing the displayed information to the information that is actually needed in the positioning step, the respective image becomes very easy to understand and intuitive to interpret, as will be especially clear from an exemplary embodiment shown below.

It is noted that a similar assisted instrument guiding means is described in U.S. patent application U.S. 61/075,467 assigned to the present assignee, for use with targeting of a target with an elongate instrument, which is included herein in its entirety by reference.

Preferably, the assisted instrument guiding means further comprises assisted instrument directing means for generating and displaying an image allowing a user to assess to which extent a plane defined by said instrument is aligned with the planned section plane. If the instrument defines a flat plane, for example by a number of straight parallel elongate members, alignment of the plane defined by said instrument with a planned section plane means that the respective two planes coincide. In this case, the image generated by said assisted instrument directing means can for example be a two-dimensional image displaying a projection of a portion remote from and parallel to said leading edge of the instrument onto a plane perpendicular to a vector defining a predetermined insertion direction.

In a preferred embodiment, the assisted instrument guiding means is further adapted to generate an image corresponding to a view of a virtual camera placed at the leading edge of the instrument. Preferably, the assisted instrument guiding means is further adapted to display medical images of predetermined objects, in particular, but not limited to, blood vessels, tumors, bony structures and organs. Accordingly, upon insertion of the instrument, the user can confirm that such objects are reliably avoided.

In an alternative embodiment, the computer assisted instrument guiding means may comprise means for projecting light signals onto the soft tissue body part indicating where the instrument is to be positioned and/or how it is to be directed for proper insertion. In this example, the instrument need not be a navigated instrument.

In a further alternative embodiment the computer assisted instrument guiding means may comprise a robot suitable for manipulating the instrument for proper insertion.

In yet another embodiment, the computer assisted instrument guiding means may be based on augmented reality. In augmented reality, real world and computer-generated image data are combined, such that computer graphics objects may be blended into real images. For example, real world video images can be digitally processed to be augmented by the addition of computer-generated graphics. In one embodiment, images of the real soft tissue body part may be augmented with graphics indicating how the instrument is to be positioned and/or how it is to be directed for proper insertion. In an alternative embodiment, a surgeon may wear augmented reality glasses in which the real world field of view as seen through the glasses may be augmented with graphics indicating how the instrument is to be directed for proper insertion. In this embodiment too, the instrument need not be a navigated instrument.

In a preferred embodiment, the system further comprises means for tracking said navigated instrument such as to continuously locate the position and orientation thereof in a tracking coordinate system. Herein, the tracking means may be configured for tracking said navigated instrument based on signals received from optical and/or electro-magnetic and/or ultra sound means. However, other types of tracking means are conceivable, such as mechanical tracking or fiber-optics tracking. Further, the system is preferably adapted to register said tracking coordinate system with a coordinate system of an intraoperative medical image of said body part. Also, the system preferably comprises means for registering a planning medical image containing the planned section plane with the intraoperative image. In a preferred embodiment, there will thus be two registering steps, namely a first registering step of registering the planning medical image with the intraoperative image and second step of registering the intraoperative image with the tracking coordinate system. However, the first and second registering steps may be combined in a single registering step. Due to this two-step registering, the location of the planned section plane in the tracking coordinate system can be obtained.

With regard to the first registering step, in some cases it cannot be expected that the planning medical image and the intraoperative medical image reflect the same motion state of the organ under surgery, since the organ has been exposed in between the acquisition of the two images. Accordingly, registering the planning medical image with the intraoperative medical image will take some deformation between the images into account.

As regards the second registering step, in one embodiment, registering the intraoperative image with the tracking coordinate system is achieved by locating the medical imaging apparatus within the tracking coordinate system and using information about the spatial relationship between the medical imaging apparatus and the medical image. For this purpose, markers could be attached to the medical imaging apparatus that can be located by the tracking system. However, this registration is only valid as long as the patient and in particular the organ under surgery does not move after taking the intraoperative medical image. Subsequent movement of the body part under surgery could be detected and accounted for by using navigation aids, such as fiducials, to be provided on or inserted into the body part.

While the coordinate system of the intraoperative medical image can be registered with the tracking coordinate system, due to soft tissue motion, in some cases the motion state of the intraoperative medical image of the soft tissue body part need not coincide with the actual motion state thereof upon insertion of the instrument. According to one embodiment of the invention, such soft tissue motion can be accounted for during an initial registering step and optionally also during consecutive registering steps for real-time compensation of soft tissue motion. In one embodiment, the navigation aids are inserted prior to taking the intraoperative medical images. During surgery the navigation aid may then be tracked during a time interval in which the navigation aids may move along with the body part due to soft tissue motion. A motion state during this interval may be determined in which the positions of the navigation aids coincide best with their positions in the intraoperative medical image. Then, the registration of the intraoperative image with the tracking coordinate system may be performed based on the tracked position of the navigation aids in said determined motion state. The rationale behind this embodiment is that a deviation of the motion state of the body part from the motion state in which the intraoperative medical image was taken is reflected in a deviation of the tracked positions of the navigation aids from their positions in the intraoperative medical image. Determining the motion state in which the positions of the navigation aids coincide best with their positions in the intraoperative medical image thus allows to identify a motion state that is very close to the motion state of the body part upon taking the intraoperative medical image.

If navigation aids are applied prior to taking the intraoperative medical image, the system may be configured for repeatedly determining and displaying a value indicating how well the current positions of the navigation aids in the tracking coordinate system correspond with their positions in the intraoperative medical image. An example of such a value, called fiducial registration error (FRE) is described in more detail in the co-pending patent application U.S. 61/075,467 and the references cited therein. If the current motion state of the body part leads to a small FRE, this indicates that the motion state is similar to the one in which the registration of the intraoperative image with the tracking coordinate system has been performed, which means that in this instant, the problems due to soft tissue motion are less pronounced. In other words, the FRE can serve as a confidence value that the registration of the intraoperative image and tracking coordinate system currently is valid.

Additionally or alternatively, the system may comprise means for compensating the motion of the soft tissue, said means being configured to calculate the current position of the planned section plane based on information of the motion state of the body part. Herein, the information of the motion state of the body part may be represented by the positions of navigation aids attached to and/or inserted to the body part, and the calculation may be based on a deformation model of the body part.

Note that navigation aids are just one possible means of observing the motion state of the body part. In an alternative embodiment conceived by the inventors, a tracked 3D ultrasound imaging apparatus could be used to continuously acquire medical images of characteristic land marks of the organ under surgery, such as its vessel tree, which could then be matched in real time with the corresponding landmarks planning medical image. With this real time approach, all soft tissue motion during the intervention can be accounted for in a very powerful manner.

### BRIEF DESCRIPTION OF THE FIGURES

The accompanying drawings illustrate embodiments of the invention and, together with the description, serve to explain the principles of embodiments of the invention.

FIG. 1 is a schematic diagram illustrating the steps performed in planning a section plane and inserting the instrument of the invention such as to define at least a portion of a section plane coinciding with said planned section plane,

FIG. 2 is a schematic perspective view of an embodiment of a navigated instrument,

FIG. 3 is a schematic perspective view illustrating the function of the positioning assisting 3 means according to one embodiment of the present invention,

FIG. 4 is a screenshot of an image generated and displayed by positioning assisting means according to an embodiment of the invention,

FIG. 5 is a schematic perspective diagram illustrating the function of an assisted instrument directing means according to an embodiment of the present invention,

FIG. 6 is a screenshot of an image generated and displayed by an assisted instrument directing means according to an embodiment of the present invention,

FIG. 7 is a schematic perspective image illustrating the function of the assisted instrument insertion-guiding means,

FIG. 8 is a screenshot of a virtual image generated and displayed by the assisted instrument insertion-guiding means of FIG. 7,

FIG. 9 shows a number of schematic views of a cutting tool for use with the system and method according to one embodiment of the present invention.

FIG. 10 shows two schematic side views of a laparoscopic cutting tool for use with the system and method of the invention.

### DESCRIPTION OF THE PREFERRED EMBODIMENT

For the purposes of promoting an understanding of the principles of the invention, reference will now be made to the preferred embodiments illustrated in the drawings and specific language will be used to describe the same. It will nevertheless be understood that no limitation of the scope of the invention is thereby intended, such alterations and further modifications in the illustrated device and method and such further applications of the principles of the invention as illustrated therein being contemplated as would normally occur now or in the future to one skilled in the art to which the invention relates.

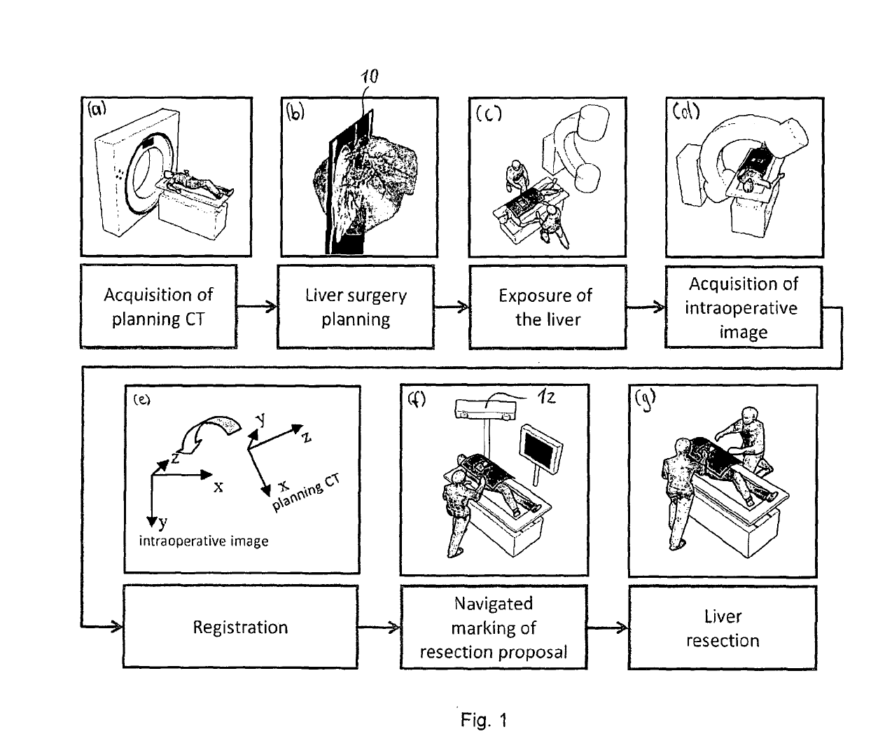

In FIG. 1, the workflow of computer assisted liver surgery is schematically summarized, in which the method and system of the invention can be employed. By way of example only, the intervention is considered to be the excision of a tumor in a human body's liver. However, it is to be understood that the system and method of the invention is by no means limited to this application and can be employed for any type of surgery in soft tissue.

In a first step, schematically shown in (a) of FIG. 1, a medical image for planning the surgery is acquired. In the present example, the medical image is a CT image, but other types of medical images, such as NMR-images could also be used. This planning medical image could be acquired a few days or even weeks prior to the actual surgery, depending on the type of surgery to be performed.

The medical image data acquired in step (a) can then be used in a planning software for calculating a resection proposal, as indicated in panel (b) of FIG. 1. For example, the planning software may construct a three-dimensional model of the patient's anatomy from the medical image data acquired in step (a). From this model, an appropriate resection plane proposal can be calculated, taking account of for example the volume of the target organ, the position of the tumor, the position of risk structures such as large vessels that have to be avoided, the blood supply and drainage, the volume of the part of the liver remaining with the patient etc. Also, the appropriate section plane 10 will be such that it can ensure that the whole tumor will be resected, while as much healthy tissue as possible remains with the patient. The function of such surgery planning software is described in Fischer L, Hoffmann K, Neumann J O, Schöbinger M, Grenacher L, Raeleff B, Friess H, Meinzer H P, Büchler M W, Schmidt J, Schemmer P; “The Impact of Virtual Operation Planning on Liver Surgery”; Imaging Decisions 2007; 1:39-44, which is incorporated herein by reference.

In step (c) the actual liver surgery starts with exposure of the liver. In step (d) of FIG. 1 an intraoperative CT image of the patient's body part (i.e. the abdomen) containing the liver is taken. As above, note that in the general framework of the invention different types of medical imaging could be used, such as NMR imaging and ultrasound imaging.

In step (e) the coordinate system of the planning CT acquired in step (a) and the coordinate system of the intraoperative CT acquired in step (d) are registered with each other. Accordingly, the planned section plane determined in step (b) can be defined in the intraoperative image coordinate system.

Registering the intraoperative image and the planning image is a first registering step performed in the exemplary work flow of FIG. 1. This registration of the two images amounts to a matching of the images with each other. Note that this registering will generally not involve a rigid coordinate transformation only, since in the present example after the patient has been opened, the shape or motion state of the liver will be different from the shape when taking the planning medical image. Instead, this first registering step will need some type of coordinate transformation accounting for deformation of the body part. After this first registering step, the planned section plane can be transferred from the planning image to the intraoperative image. The first registering step could for example be based on a registering of the respective vessel trees of the respective images.

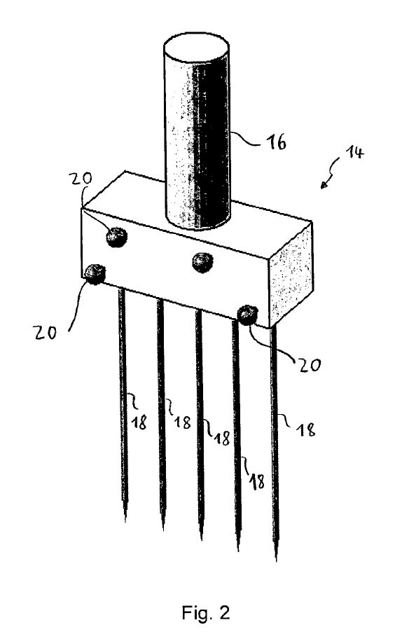

Next, in step (f), an instrument is inserted into the liver such as to define at least a portion of a section plane therein. In the embodiment shown, the instrument is a navigated marking tool 14, schematically shown in FIG. 2 and described in further detail below. However, this is only one illustrative example of a navigated instrument, and in the same manner a navigated cutting tool or a navigated jig or any other navigated tool could be used that is suitable to define at least a portion of a section plane with respect to the soft tissue body part.

To allow for navigation of the navigated instrument, a tracking system 12 is provided which allows tracking of the navigated instrument in a tracking coordinate system. Also, in order to guide the navigated instrument such as to find the planned section plane, the intraoperative medical image needs to be registered with the tracking coordinate system. This is the second registration step used in the work flow of FIG. 1.

There are different strategies conceivable to register the intraoperative image coordinate system with the tracking coordinate system, three of which shall be briefly discussed for illustrative purposes only:

A first variant would be based on a tracked medical imaging apparatus. Namely, if the medical imaging apparatus used in step (d) of FIG. 1 itself is tracked in the tracking coordinate system, the location of the intraoperative medical image acquired thereby can also be determined in the tracking coordinate system or in other words, be registered with the tracking coordinate system. This variant has the advantage that it is comparatively easy and fast to employ. However, this variant of course only works if it can be ensured that after taking the intraoperative medical image, the patient is not moved with respect to the tracking coordinate system or only moved in a way that can be accounted for.

In a second variant, markers such as fiducial needles could be attached to or inserted into the liver prior to taking the intraoperative medical image, i.e. between steps (c) and (d) of FIG. 1.

In a preferred embodiment fiducial needles (not shown) are inserted into the liver, such that their tips will lie within the liver and in the vicinity of the tumor to be excised. The fiducial needles preferably have a needle-shaped body with a rotationally symmetric elongate portion serving as a marking portion for tracking. Suitable embodiments of such fiducial needles are described in EP 1632194A1. Custom-designed silicon patches may be used to affix the fiducial needles to the skin of the patient and to prevent them from slipping out. Alternatively, the fiducial needles are fixed in the liver. As has been demonstrated in the article “Soft Tissue Navigation Using Needle-Shaped Markers: Evaluation of Navigation Aid Tracking Accuracy and CT Registration”, in proceedings of SPIE Medical Imaging 2007: Visualization, Image-Guided Procedures and Display, K. R. Cleary and M. I. Miga, editors 65026 (12 pages), February 2007, L. Maier-Hein, D. Maleike, J. Neuhaus, A. Franz, I. Wolf and H.-P. Meinzer, such fiducial needles can be constructed precisely to obtain sub-millimeter tracking accuracy.

Since the fiducial needles can be tracked by the tracking system 12 and are also visible in the intraoperative medical image, the latter can be registered with the tracking coordinate system in a way known per se from prior art. Note that such fiducial needles or other types of markers have the additional advantage that they allow to notice and to a certain extend correct for deformation or other types of motion of the body part after the intraoperative medical image has been taken, in a way explained in more detail in the co-pending application U.S. 61/075,467.

A third and very elegant exemplary method of registration conceived by the inventors is a method in which the first and second registration steps are continuously or repeatedly performed in real time. For example, instead of taking a single intraoperative image as indicated in panel (d) it is suggested to repeatedly or continuously take intraoperative images in real time, for example using a tracked 3D ultrasound imaging apparatus. Using appropriate software, certain landmarks or structures of the liver such as the vessel system could be identified and registered with the planning medical image. This way, the motion or deformation state of the organ during surgery can be fully and elegantly accounted for. Note that this third variant can be regarded as an extension of the first variant, in which the first and second registering are repeatedly performed.

As mentioned above, in panel (f) of the embodiment of FIG. 1 the planned section plane 10 or at least a portion thereof is marked using a navigated marking tool 14 schematically shown in FIG. 2, where it is again understood that a marking tool is only one example of a navigated instrument for use in the invention. With reference to FIG. 2, the marking tool 14 comprises a handle 16 for holding by the user and a number of parallel elongate needle-like elements 18. The elongate needle-like elements 18 define a common plane. Thus, when the needle-like elongate elements 18 are inserted into the liver, this allows to define a section plane within the soft tissue body part. Also, as further seen in FIG. 2, markers 20 are attached to the marking tool 14, such that the position and orientation thereof can be determined in the tracking coordinate system by means of tracking system 12. Accordingly, the marking tool 14 is a navigated marking tool.

With further reference to panel (f), the navigated marking tool 14 is inserted to the liver such that the section plane defined thereby coincides with the planned section plane 10 determined in step (b). For this purpose, computer assisted instrument guiding means to assist or enable guiding the instrument 14 upon insertion into the liver are provided. Examples of such computer assisted instrument guiding means are described below. An example of computer assisted guiding means “enabling” guiding the instruments could for example be a robot for inserting the same.

Also in the step of panel (f), the section plane defined by navigated marking instrument 14 is marked using appropriate marking means. In the present embodiment, the marking means comprise means for coagulating the tissue surrounding the needle-like elongate elements 18 by applying a suitable HF electrical field thereto. Due to coagulation, the color of the surrounding tissue changes, such that the section plane defined upon insertion of the navigated marking tool 14 is permanently visible in the tissue, even after removing the navigated instrument 14.

Accordingly, in step (g) of FIG. 1, the liver resection can be carried out by cutting the liver tissue along the coagulated pattern visible in the liver tissue, which in turn represents the planned section plane 10. In particular, since the tissue itself is marked, subsequent soft-tissue motion or topology change after the liver has been cut do not cause any problems at all, since the surgeon simply has to follow the markings directly provided in the liver tissue. This is a remarkable advantage over prior art computer assisted surgery, where motion and topology changes make the orientation very difficult, such that it is extremely difficult to transfer the planned section plane 10 to the actual organ under surgery. In the context of the present invention, however, a two-dimensional section plane with respect to the organ under surgery can be permanently defined upon a single step of navigated insertion of an appropriate instrument such as the marking tool 14 of FIG. 2 and a consecutive marking step.

While in the embodiment shown in FIG. 2 the needle-like elongate elements 18 are straight, such that a flat section plane can be defined, the elongate members 18 could also be curved for defining a curved section plane. In a preferred embodiment, the system for computer assisted surgery would comprise a set of different navigated marking tools having different sizes and curvages.

Also, while in the present embodiment the marking means are configured for marking the tissue by coagulation, in principle marking by any type of denaturation which has a visible effect on the tissue can be used, as can different types of means for causing such denaturation including monopolar or bipolar electric fields, heat, coldness, chemical agents such as etchants etc. Marking the tissue by coagulation or cauterization however has the further special technical effect that bleeding upon insertion and/or subsequent cutting can be limited or even avoided. Also, marking the tissue by coagulation or cauterization has the advantage that in case a tumor has been accidentally hurt by the navigated marking tool 14, tumor seeding may be prevented by coagulation or cauterization in a way per se known from tumor ablation.

In an alternative embodiment the marking means of the navigated instrument may comprise means for applying color to the tissue or means for inserting marking objects into the tissue. In principle, any conceivable type of marking can be used that allows to identify the section plane after the navigated instrument is removed.

In an alternative embodiment the navigated instrument may be a jig defining at least a portion of a section plane along which the tissue can be cut while the jig remains inserted in the soft tissue body part. In this case, the navigated instrument itself is the marker.

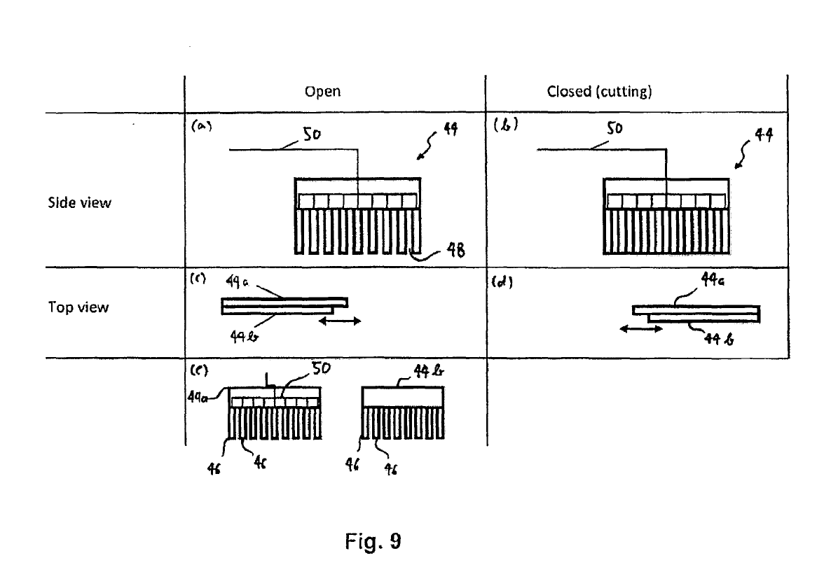

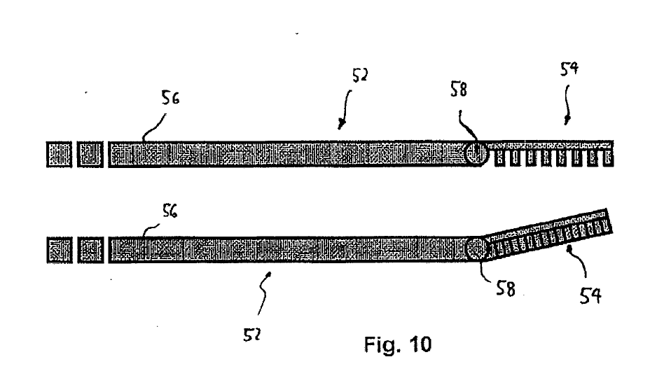

In a further preferred embodiment, the navigated tool can be a cutting tool, which may cut the tissue after insertion thereof. An example of such a navigated cutting tool is described below with reference to FIGS. 9 and 10.

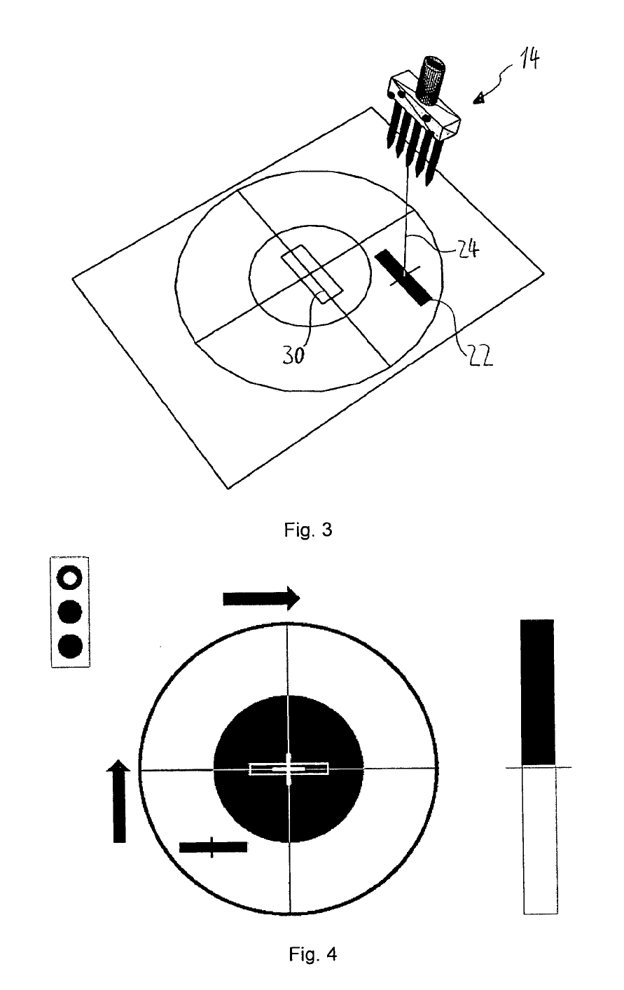

As becomes apparent from the work flow of FIG. 1, according to the invention the transfer of the planned section plane 10 to the patient's soft tissue body part essentially amounts to a single step of inserting the instrument 14 to the soft tissue body part such that the section plane defined by the inserted instrument 14 coincides with the planned section plane 10. In some embodiments, a sequence of insertion steps may be employed each defining a portion of a section plane, where the instrument is pushed forward in a number of consecutive steps. In order to assist guiding the instrument 14 upon insertion into the body part, according to the invention computer assisted instrument guiding means are provided, which will be described next with reference to FIG. 3 to 7.

Inserting the navigated instrument 14 into the soft tissue body part comprises three crucial steps: The first is to position the leading edge of the navigated instrument 14 properly on the surface of the soft tissue body part. The second step is to properly direct the instrument such that the plane defined by the instrument coincides with the planned section plane 10. In other words, this second or directing step is a step of tilting the instrument until the instrument is aligned with the planned section plane. In a third step, the instrument is inserted into the soft tissue body part up to a predetermined depth if the planned section plane and the corresponding navigated instrument are curved, the third step also comprises guiding the instrument along a curved insertion path. In any case, the instrument has to be guided such as to not deviate from the planned section plane.

In a preferred embodiment of the present invention, the computer assisted instrument guiding means generates and displays appropriate images that support the user in carrying out each of the three sub-steps of insertion of the instrument. This computer assisted guiding is similar to the guiding described in the co-pending application U.S. 61/075,467, with regard to targeting of a target such as a tumor with an elongate instrument, such as a biopsy needle. However, the inventors have confirmed that such computer assisted guiding means co-invented by some of the present inventors for use with targeting of a target with an elongate instrument can also be very successfully used with the insertion of the navigated instrument of the present invention.

FIG. 4 shows a screenshot of an image generated and displayed by positioning assisting means according to an embodiment of the present invention. In this image, a projection 22 of a leading edge of the navigated instrument 14 onto the surface of the body part is displayed. Herein, the “leading edge” is the line connecting the tips of the needles 18 of the navigated instrument 14 and the projection is a projection along a vector 24 defining a predetermined insertion direction, as is illustrated by the schematic perspective view of FIG. 3.

Also in the exemplary screenshot of FIG. 4 a depth indicator 26 is displayed. The depth indicator 26 is a bar diagram representing the distance between the leading edge of the instrument 14 and the envisaged insertion depth for defining the planned section plane 10 or a respective portion thereof. If the bar of the depth indicator 26 has reached a center line 28, this means that the leading edge of the instrument 14 has reached the surface of the organ under surgery, for example the liver. Also shown in FIG. 4 is a box 30 including the intersection line of the surface of the body part and the planned section plane 10.

Further, in FIG. 4 a signal light 32 and arrows 34 and 36 are displayed, the function of which will be explained below.

The image generated by the positioning assisting mans as displayed in FIG. 4 is meant to assist the surgeon in properly positioning the leading edge of the instrument 14 on the surface of the organ under surgery, such as the liver. When the surgeon lowers the instrument 14 to the surface of the organ, he only has to make sure that the projection 22 coincides with the box 30. The two-dimensional information displayed in FIG. 4 is the crucial information for properly positioning the instrument, while the third dimension can be accessed by the surgeon easily by noticing that the tips of the needle-like members 18 have touched the surface of the organ. Also, this third dimension is reflected by the depth indicator 26. This rather abstract way of separately displaying the critical two dimensions has been found to greatly assist the surgeon in properly positioning the instrument. Positioning is further assisted by guiding arrows 34, 36 indicating the surgeon how the instrument has to be moved such as to be positioned properly. Once the instrument 14 has been positioned at the surface of the organ with the predetermined precision, this is indicated by the signal 32 and the positioning step is completed.

In the next step, the instrument 14 shall be aligned with the planned section plane 14. This is assisted by assisted instrument directing means for generating and displaying an image allowing a user to assess to which extent a plane defined by the instrument 14 is aligned with the planned section plane 10.

If the planned section plane 10 and the plane defined by the set of elongate elements 18 is a flat plane, this alignment amounts to tilting the instrument 14 such that the angle between the two planes becomes zero. If the planes are curved planes, the alignment becomes somewhat more complicated. In that case, the tangential plane at the leading edge of the instrument would have to be aligned with the tangential plane to the planned section plane 10 at the intersection with the surface of the organ.

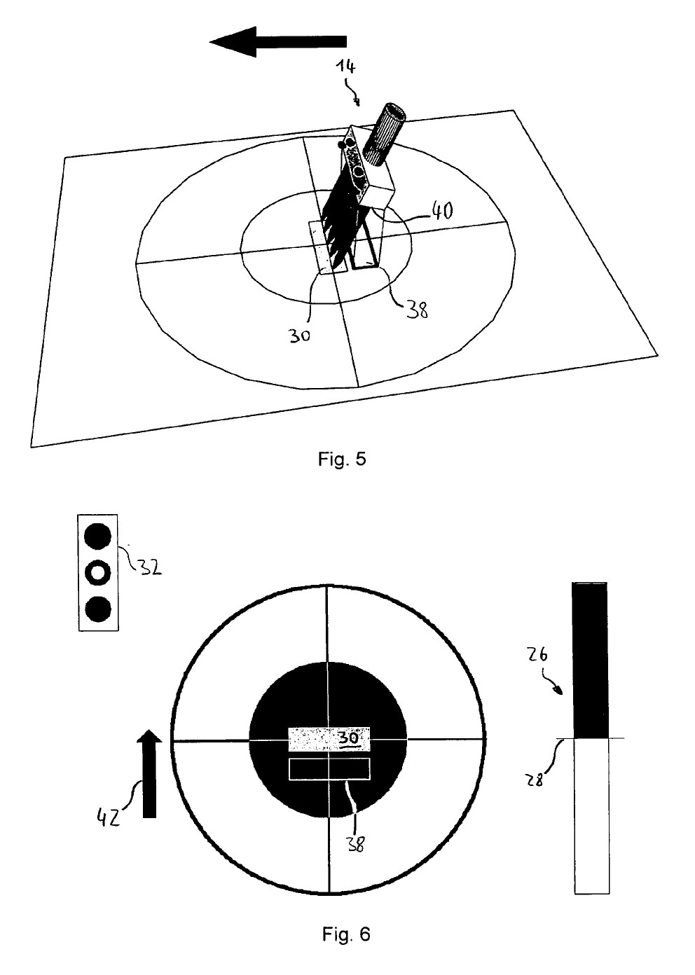

With reference to FIGS. 5 and 6, the case of flat planes is discussed. Herein, FIG. 6 shows an image generated and displayed by assisted instrument directing means. The image of FIG. 6 is very similar to the image of FIG. 4. However, this time a projection 38 of a portion 40 remote from and parallel to said leading edge of the instrument 14 onto a plane perpendicular to the above mentioned vector 24 defining a predetermined insertion direction is displayed as is illustrated in the schematic perspective view of FIG. 5. While monitoring the image displayed in FIG. 6, the user has to tilt the instrument 14 such that the projection 38 coincides with box 30, indicating that the angle between the planned section plane 30 and the plane defined by the elongate needle-like elements 18 has become zero. Again, the necessary tilting is facilitated by an arrow 42 indicating how the instrument 14 needs to be tilted. If the instrument 14 is sufficiently aligned with the planned section plane, this is indicated by signal light 32 also shown in FIG. 6.

Note that the positioning assisting means and the assisted instrument directing means shown with reference to FIGS. 4 and 6 are only exemplary embodiments, which could be modified in various ways. Irrespectively of the specific way the generated images look, it has been found to be helpful to provide two-dimensional abstract images specifically adapted for assisting the user in positioning and/or directing the instrument. In particular, it has been found to be helpful if the two dimensions represented by the image correspond with the two crucial dimensions of the three-dimensional movement to be performed, be it positioning or directing.

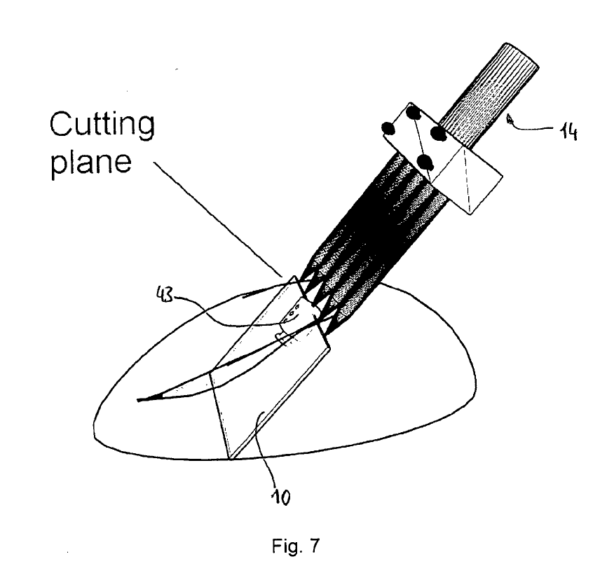

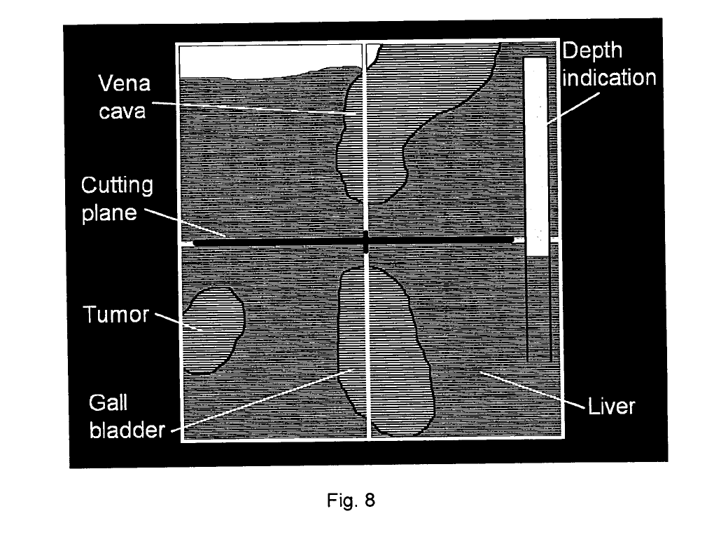

In the third step, the properly positioned and directed instrument 14 is inserted into the soft tissue body part along the planned section plane 10. This insertion is facilitated by an assisted instrument insertion guiding means adapted to generate an image corresponding to a view of a virtual camera 43 placed at the leading edge of the instrument 14. The concept of the virtual camera 43 is schematically illustrated in FIG. 7, while a view provided by such virtual camera 43 is shown in FIG. 8. As is seen in FIG. 8, the cutting plane 10 is also inserted into the virtual camera image. By providing the virtual camera image, the surgeon can be sure not to accidentally hurt any risk structures like large blood vessels or tumors upon insertion. Also in FIG. 8, the depth indicator 26 is displayed indicating how far the instrument 14 has to be inserted into the soft tissue body part.

As has become apparent from the description of FIG. 3 to 8, the computer assisted guiding means greatly facilitate insertion of the navigated instrument 14 such that the section plane defined by the inserted instrument 14 coincides with the planned section plane 10.

Note that the computer assisted guiding of the navigated instrument relies on the registration of the preoperative medical image with the intraoperative medical image and the registration of the intraoperative medical image with the tracking coordinate system. As is described in more detail in the co-pending application U.S. 61/075,467 and the references cited therein, registration errors may occur due to soft tissue motion. However, these errors can be kept small and/or compensated for by means also described in more detail in the co-pending application U.S. 61/075,467 such that the details are not repeated herein. Besides instrument manipulation and movement of the patient, in many cases a main source of soft tissue motion is breathing. If fiducials are applied prior to taking the intraoperative image, the system may be configured to repeatedly determine and display a value indicating how well the current positions of the navigation aids correspond with their positions in the intraoperative medical image. An example for such a value is the so-called fiducial registration error, which is described in more detail in the co-pending application U.S. 61/075,467 and the references cited therein. If this value indicates that the current positions of the navigation aids are similar to those in the intraoperative medical image, it can be assumed that the current motion state is very similar to the motion state in the intraoperative image which in turn means that the registration of the intraoperative image and the tracking coordinate system is presumably very precise. Accordingly, the surgeon may observe this value and perform the insertion in a time period or consecutive time periods in which the registration is assumed to be very precise.

In addition, the system can be refined by providing means not only for detecting but actually for compensating the motion of the soft tissue, said means being configured to calculate the current position of the planned section plane based on information of the motion state of the body part, which may be represented by the position of the navigation aids, where the calculation may for example be based on a deformation model of the body part.

With reference to FIGS. 9 and 10 alternative examples of navigated instruments for use with the system of the present invention are shown. In FIG. 9, side and top views of a navigated instrument 44 in an open state (left column) and a closed state (right column) are shown. The navigated instrument is a navigated cutting tool which when inserted into a body part defines at least a portion of a section plane and which while inserted may cut the tissue along the section plane.

The navigated instrument 44 is comprised of first and second members 44a and 44b which are shown separately in FIG. 9(e). Each of said first and second members 44a, 44b has a comb-like structure with a set of substantially parallel blade like elements 46 spaced at equal intervals. In the assembled state the first and second members 44a, 44b can be moved with respect to each other between a first or open position shown in panels (a) and (c) and a second or closed position, shown in panels (b) and (d) of FIG. 9. In the first or open position, the blade-like elements 46 of the first member 44a are congruent with the blade-like elements 46 of the second element 44b, leaving free spaces 48 in between when viewed from a direction perpendicular to the direction of relative motion of the first and second members 44a, 44b. Hence, this position is called the “open position”.

In the second or closed position, blade-like elements 46 of the first member 44a are located between adjacent blade-like elements 46 of the second member 44b, when viewed in a direction perpendicular to the direction of relative motion as is seen in FIG. 9(b). For obvious reasons, the second position is also called the closed position.

Finally, in the embodiment of FIG. 9, the blade-like elements 46 of the first member 44a are connected with conductors 50 which in turn are connected with an electro-surgical generator (not shown) such that an appropriate voltage, frequency or waveform can be applied to the blade-like elements 46.

Next, the operation of the navigated instrument 44 will be explained:

The navigated instrument 44 in the open position can be inserted into a living object's body part in just the same way as described above with reference to FIG. 1(f) in the context of the navigated instrument 14 of FIG. 2. In particular, the same computer assisted instrument guiding means as described with reference to FIG. 3 to 8 could be used. After the instrument 44 is properly inserted to define a section plane corresponding to the planned section plane 10, the elongate elements 46 of the first member 44a of the instrument 44 are powered by the electro-surgical generator (not shown) such as to coagulate the tissue surrounding each of the blade-like elements 46 as well as the tissue in the spaces 48 between adjacent blade-like elements 46. For example, the tissue may be coagulated within an area equal or less than 0.5 to 1 cm away from one of the blade-like elements 46. By the coagulation, bleeding caused by insertion of the element 44 into the tissue will be stopped.

Next, first and second members 44a, 44b are moved from the open position (see FIG. 9 (a), (c)) to the closed position (see FIG. 9 (b), (d)), thereby cutting the coagulated tissue in the spaces (48) between adjacent elongate blade-like elements 46. Accordingly, the tissue is completely cut along the section plane defined by the instrument 44 upon insertion. At the same time, due to the previous coagulation step, no bleeding will occur upon cutting the tissue. Also, if accidentally a tumor should be cut, tumor seeding can reliably be prevented due to the coagulation step. The navigated cutting instrument 44 shown in FIG. 9 thus allows for a safe and fast cutting of tissue. Moreover, it is very easy to use and prevents bleeding and tumor seeding.

While the instrument 44 shown in FIG. 9 is designed for use in open surgery, the same principle can be applied in a laparoscopic instrument as well, as is shown at reference sign 52 in FIG. 10. The instrument 52 has a front portion 54 which is similar to the cutting tool 44 shown in FIG. 9 and is hingedely connected to an elongate instrument body 56 by a hinge 58. The distal end of instrument body 56 is connected with a handle for guiding the instrument (not shown) and an electro-surgical generator (not shown).

While the above preferred exemplary embodiments have been described with reference to navigated instruments, depending on the computer assisted instrument guiding means employed the instrument itself need not be navigated. For example, means could be provided for projecting light signals onto the soft tissue body part indicating how the non-navigated instrument is to be positioned and/or directed. A further example comprising computer assisted instrument guiding means which would not necessarily employ a navigated instrument is the guiding means based on augmented reality mentioned above.

Although preferred exemplary embodiments are shown and specified in detail in the drawings and the previous specification, these should be viewed as purely exemplary and not as limiting the invention. It is noted in this regard that only the preferred exemplary embodiments are shown and specified, and all variations and modifications should be protected that presently or in the future fall within the scope of protection of the invention.

### LIST OF REFERENCE SIGNS

10 planned section plane,
12 tracking system,
14 navigated marking tool,
16 handle,
18 needle-like element,
20 marker,
22 projection of leading edge of instrument 14,
24 vector defining insertion direction,
26 depth indicator,
28 center line of depth indicator,
30 box including section of planned section plane with surface of body part under treatment,
32 signal light
34, 36 arrows,
38 projection of remote portion 40 of instrument 14,
40 remote portion of instrument 14,
42 arrow,
43 virtual camera,
44 navigated cutting tool,
44a first member of navigated cutting tool 44,
44b second member of navigated cutting tool 44,
46 blade-like element,
48 space between adjacent blade-like elements 46,
50 conductor,
52 laparoscopic navigated cutting tool,
54 front portion of laparoscopic cutting tool 52,
56 instrument body,
58 hinge

1. A system for computer assisted surgery in soft tissue, comprising:
an instrument configured to be inserted into a living object's soft tissue body part, wherein said instrument when inserted into the body part is configured to define at least a portion of a section plane with respect to said soft tissue body part, and
a computer assisted instrument guiding means to assist or enable guiding the instrument upon insertion into the body part such that the section plane defined by said inserted instrument coincides with a planned section plane (10).

2. The system of claim 1, wherein said instrument comprises a set of elongate members (18, 46) configured for being simultaneously inserted into said soft tissue body part.

3. The system of claim 1, wherein the number of elongate members is three or more, preferably five or more.

4. The system of claim 1, wherein the distance between adjacent elongate members is between 0.5 cm and 3.5 cm.

5. The system of claim 1, wherein said elongate members comprise a set of substantially parallel needle-like or blade-like elements.

6. The system of claim 1, wherein said navigated instrument is provided with marking means for marking the section plane or portions thereof within the soft tissue body part.

7. The system of claim 6, wherein said marking means of said instrument comprises one or more of the following: denaturation means for marking the section plane by denaturation, in particular cauterisation or coagulation, colour marking means for marking the section plane by means of applying colour to the tissue, or marking objects insertion means for inserting marking objects.

8. The system of claim 7, wherein said instrument when inserted into the soft tissue body part, is configured for coagulating or cauterising the tissue in the vicinity of each elongate member and/or in between adjacent elongate members.

9. The system of claim 2, wherein said instrument is a navigated cutting tool configured for coagulating or cauterising the tissue in the vicinity of each elongate member or between adjacent elongate members and for cutting tissue between at least some adjacent ones of said elongate members when inserted into the soft tissue body part.

10. The system of claim 9, wherein said instrument comprises an additional set of blade members configured for cutting the tissue between said elongate members after insertion into the soft tissue body part.

11. The system of claim 10, wherein said instrument comprises first and second members,
each of said first and second members comprising a set of substantially parallel bladelike elements,
said first and second members being moveable relative to each other to a first position, in which the bladelike elements of the first member are substantially congruent with corresponding bladelike elements of the second member, said corresponding bladelike elements in said congruent state forming said elongate members, and
a second position, in which, when viewed from a direction perpendicular to the direction of relative motion, bladelike elements of the first member are located substantially between adjacent bladelike elements of said second member.

12. The system of to claim 11, wherein said instrument is a laparoscopic instrument

13. The system of claim 1, wherein said instrument is configured for cutting the tissue between adjacent elongate members by electro surgery.

14. The system of claim 10, configured for applying an electrosurgical cutting voltage to some or all of said blade members.

15. The system of claim 1, wherein said instrument is a jig which defines a section plane along which the tissue can be cut while the jig remains inserted in the soft tissue body part.

16. The system of claim 1, wherein said assisted instrument guiding means comprises positioning assisting means for assisting a user to position the instrument at the soft tissue body part prior to insertion,
said positioning assisting means being configured to generate and display an image specifically for allowing a user to assess how the leading edge of the instrument has to be moved in order to approach the intersection line of the surface of the body part and the planned section plane.

17. The system of claim 16, wherein the image generated by said positioning assisting means represents a relative position between said intersection line and a projection of said leading edge of said instrument onto the surface of said body part.

18. The system of claim 17, wherein said projection is a projection along a vector defining a predetermined insertion direction.

19. The system of claim 1, wherein said assisted instrument guiding means comprises assisted instrument directing means for generating and displaying an image specifically for allowing a user to asses to which extent a plane defined by said instrument is aligned with the planned section plane.

20. The system of claim 19, wherein the plane defined by the said instrument is a flat plane and the image generated by said assisted instrument directing means is a two-dimensional image displaying a projection of a portion remote from and parallel to said leading edge of the instrument onto a plane perpendicular to a vector defining a predetermined insertion direction.

21. The system of claim 1, wherein said assisted instrument guiding means is configured to generate an image corresponding to a view of a virtual camera placed at the leading edge of the instrument.

22. The system of claim 21, wherein said assisted instrument guiding means is further configured to display medical images of predetermined objects, in particular, but not limited to, blood vessels, tumors, bony structures and organs.

23. The system of claim 1, further comprising tracking means for tracking said instrument such as to continuously locate the position and orientation of said instrument in a tracking coordinate system.

24. The system of claim 23, wherein said tracking means are configured for tracking said instrument based on signals received from optical and/or electromagnetic and/or ultra sound means.

25. The system of claim 23, further configured to register said tracking coordinate system with a coordinate system of an intraoperative medical image of said body part.

26. The system of claim 25, further configured to register said intraoperative medical image with a preoperative medical image containing the planned section plane.

27. The system of claim 25, further comprising navigation aids, such as fiducials, to be provided on or inserted to the body part.

28. The system of claim 27, wherein said navigation aids comprise a needle-shaped body having an elongate portion serving as a marker.

29. The system of claim 27, further configured to track said navigation aids during a time interval during which the navigation aids are allowed to move along with the body part due to soft tissue motion, and to determine a motion state of the body part in which the positions of the navigation aids coincide best with their positions in the intraoperative medical image.

30. The system of claim 27, further configured to repeatedly determine and display a value indicating how well the current positions of the navigation aids correspond with their positions in said intraoperative medical image.

31. The system of claim 1, further comprising means for compensating the motion of the soft tissue, said means being configured to calculate a current position of the planned section plane based on information of the motion state of the body part.

32. The system of claim 31, wherein said information of the motion state of the body part is represented by the positions of navigation aids attached to and/or inserted to the body part, and the calculation is based on a deformation model of the body part.

33. The system of claim 1, wherein the computer assisted instrument guiding means comprise means for projecting light signals onto the soft tissue body part indicating how the instrument is to be positioned and/or directed.

34. The system of claim 1, wherein the computer assisted instrument guiding means comprise a robot for manipulating the navigated instrument.

35. The system of claim 1, wherein the computer assisted instrument guiding means comprise means for augmenting video images of the operational site or a view through glasses worn by a surgeon by the addition of computer generated graphic indicating how the instrument is to be positioning and/or directed.

36. An instrument configured to be inserted into a living object's soft tissue body part, which when inserted into the body part is configured to define at least a portion of a section plane with respect to said soft tissue body part, wherein said instrument is provided with means allowing for a computer assisted instrument guiding of the instrument upon insertion into the body part.

37. The instrument of claim 36, wherein said means allowing for computer assisted instrument guiding comprise markers configured for being tracked by a tracking system.

38. The instrument of claim 36, wherein said instrument is provided with marking means for marking a section plane or portions thereof within a soft tissue body part.

39. The instrument of claim 36, wherein said instrument is a cutting tool comprising a set of elongate members configured for being simultaneously inserted into the soft tissue body part, said cutting tool being configured for coagulating and/or cauterizing the tissue in the vicinity of each elongate member and/or between adjacent elongate members and for cutting tissue between at least some adjacent ones of said elongate members when inserted into a soft tissue body part.

40. A method for computer assisted surgery in soft tissue, comprising a step of inserting a instrument into a living object's soft tissue body part, wherein said instrument when inserted into the body part is configured to define at least a portion of a section plane with respect to the soft tissue body part, wherein said
step of inserting the instrument into the body part is carried out using computer assisted instrument guiding means and such that the section plane defined by said inserted instrument coincides with a planned section plane.

41. The method of claim 40, further comprising a step of acquiring a preoperative medical planning image and a step of determining a planned section plane based on said preoperative planning image.

42. The method of claim 41 further comprising a step of marking the section plane or portions thereof within the soft tissue body part by means of said navigated instrument.

43. The method of claim 42, wherein said marking step comprises one or more of the following:
a denaturisation step of marking the tissue by denaturisation, in particular cauterisation or coagulation, a colour marking step of marking the section plane by means of applying colour to the tissue, or a marking object insertion step of inserting marking objects to the tissue.

44. The method of claim 43, wherein said instrument comprises a set of elongate members configured for being simultaneously inserted into the soft tissue body part, and wherein the marking step comprises a step of coagulating or cauterising the tissue in the vicinity of each elongate member and/or in between adjacent elongate members.

45. The method of claim 40, wherein said instrument comprises a set of elongate members configured for being simultaneously inserted into said soft tissue body part, said method comprising, after said insertion, a step of coagulating and/or cauterising the tissue in the vicinity of each elongate member and/or between adjacent elongate members and
a step of cutting tissue between at least some adjacent ones of said elongate members inserted into the soft tissue body part.
